(19) United States
(12) Patent Application Publication
(10) Pub. No.: US 2024/0151671 A1
(43) Pub. Date: May 9, 2024

(54) Soil-powered LED Plant Moisture Meter

(71) Applicant: Devon Mark Cioffi, Ashland, OR (US)

(72) Inventor: Devon Mark Cioffi, Ashland, OR (US)

(21) Appl. No.: 18/052,914

(22) Filed: Nov. 5, 2022

(51) Int. Cl. G01N 27/04; G01N 33/24; G08B 21/20

(52) U.S. Cl. CPC G01N 27/048; G01N 27/045; G01N 33/246; G08B 21/20; G08B 5/36

(57) ABSTRACT

The present invention comprises a novel system for indicating the water saturation level of a soil medium or soil medium that contains a plant and uses the soil medium as power source and is readable from a distance. The preferred embodiment of the device comprises and generally consists of both a magnesium rod anode and copper cylinder cathode, however other embodiments may use different metals as well as different shapes and configurations for the anode and cathode. These lead to a DC to DC converter circuit running an LED light inside a waterproof housing. When the anode and cathode are inserted into the soil in one of two configurations, the brightness of the LED will correlate with the water saturation level of the soil medium that it is installed into, providing real-time passive data to the user without a need for a conventional battery power source.

### FIELD OF THE INVENTION

The present invention relates to an environmentally friendly, novel system for monitoring the water saturation level of a soil medium. More particularly the system operates without external power source and uses the soil as a conductive path between anode and cathode to generate a small charge and light an LED indicator, which dims when the soil is dry without producing harmful by-products or creating waste, instead providing plants and soil with vital nutrients. For houseplants, hobbyist gardeners, as well as large-scale agriculture and commercial plant grows and field testing.

### BACKGROUND

Plants and houseplants frequently go over or under-watered. Due to the physical location of plants or limited time, it may not be possible for plant owners to check their soil moisture frequently enough, which will negatively impact the health of the plant and the overall soil biome.

The present invention provides real-time moisture-level data. The green LED indicator is bright and noticeable enough to be seen at a good distance, even in daylight conditions. It provides constant, passive, long-distance information to plant owners and saves plant owners the time of manually checking the moisture level of their plants, and requires no batteries, only conductive soil.

Other moisture meters require user action or cannot be read at a distance. Manual meters require an operator to insert the probe into the soil and read a digital or analog gauge. Other meters use a small buoy system and cannot be read at any great distance, especially under dark conditions.

Current moisture meters on the market require too much time and effort to be of much real value. They require the user to stop what they are doing in order to take a reading, and do not save time or provide constant adequate information that can be read from a distance. They can also be highly inaccurate.

The green LED of the present apparatus can be read from a great distance under most lighting conditions. The user can view the brightness of the constant LED light in the background passively to know that a plant has been watered and not need to actively interact with any meter to get a reading

This functionality is achieved with a clean and environmentally-friendly power source that utilizes the soil which is already present in the ground or the potted plant, while creating a time-released source of magnesium oxide as a waste product from the galvanic corrosion of the magnesium rod. MgO2 is commonly used in a wide range of existing commercial fertilizers and is not the most bio-available source of magnesium for plants which won't overwhelm most plants and cause adverse reactions to the magnesium supplementation.

### BRIEF SUMMARY OF THE INVENTION

The present invention comprises two dissimilar metals, copper and magnesium, acting as cathode and anode, respectively. They are inserted into the soil in one or two configurations and an electric charge is produced via galvanic corrosion of the anode that varies in voltage and amperage with the moisture content of the soil medium as long as sufficient electrical conductivity (EC) is present in the soil. The two metals are wired into a waterproof LED housing on a stake that contains a DC-to-DC 4-pin gated oscillator circuit, stepping up the voltage to light a 3.3v green LED, visible from across the room and visible outdoors when the soil medium is watered correctly. When the soil medium dries, the electrical output of the device is reduced and the LED grows dim and eventually shuts off entirely to indicate that the soil medium has become dry and requires watering if there is a plant contained within the soil medium. The device will detect the moisture of a soil medium without a plant present, even in a field setting. The specific oxides released by the galvanic reaction of the anode and cathode of the device are beneficial for soil as well as plants contained therein and work as time-release fertilizers to further improve the health of soil and plants being monitored with the system. The system can be used in a variety of mediums other than soil in order to investigate conductive properties, as well.

### BRIEF DESCRIPTION OF THE DRAWINGS

The embodiment of the present invention is depicted as an example and is not limited by the figure of the accompanying drawings, in which like references may indicate similar aesthetic, but not functional elements and in which:

FIG. 1 illustrates an example of an exploded perspective view of a soil-powered LED plant moisture meter according to various embodiments described herein.

FIG. 2 illustrates an example of a schematic diagram for the DC to DC converter circuit needed to light the LED according to various embodiments described herein, including the required locations of the anode and cathode metals.

FIG. 3 illustrates an example of the two possible operational/setup configurations required for the device to function properly according to various embodiments described herein.

### DETAILED DESCRIPTION OF THE INVENTION

The DC-to-DC converter circuit within the LED housing will receive a measurable current and voltage via the positive and negative wires that enter the housing. This electron flow is supplied by the galvanic corrosion of the magnesium anode, where the soil that it and the copper anode is inserted into acts as a conductive solution, providing a conductive path between the two (from anode to cathode). The efficiency of this conductive path varies with soils of different quality and is contingent upon a number of factors, such as the level of salt which affects EC (electrical conductivity), the type of water used, as well as metals and present in the soil such as iron. Chlorinated “city” water will decrease the conductivity of soil as well as prematurely corrode the magnesium rod and is not recommended. Magnesium Sulfate (Epsom salt) or blood meal, which contains iron can be used to artificially inflate the soil conductivity and increase the effectiveness of the device. Both of which are also safe for plants and recommended in small amounts as common soil amendments.

The anode and cathode are either arranged side-by-side or one inside the other, with the magnesium rod anode inserted into the soil medium within the cylindrical copper cathode. The conductive properties of the soil changes with its moisture content. When the soil dries, it will act as an increasingly poor electrolyte and conductive path, and the current received by the PCB board which drives the green LED will be diminished, dimming and eventually turning off the light. The light will achieve full brightness in wet soil with around 10-20 mA of current and start to dim between 10-5 mA as the soil dries. 5 mA or less current will further dim the light until it eventually starts to turn off with less than 4 mA of current, indicating soil dryness.

The galvanic reaction of the magnesium rod anode within the soil medium next to or within the copper pipe cathode provides about 1.2v-1.5v of voltage on average and 0-20 mA of current depending upon the moisture content of the soil medium, altering the brightness of the LED when connected through the DC-DC converter circuit inside the waterproof housing.

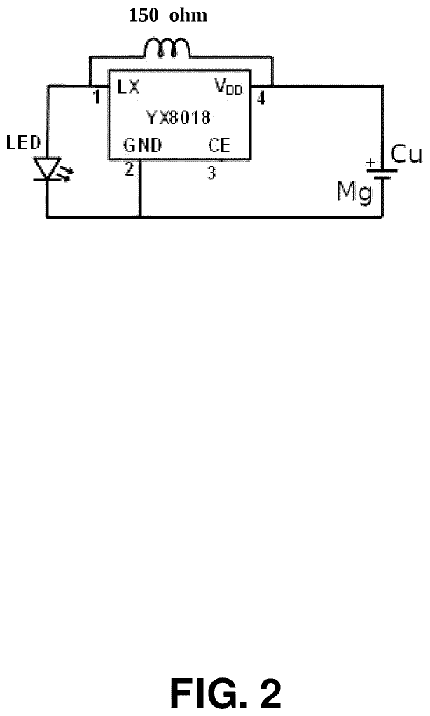

As described in FIG. 2, the 3.3v LED is illuminated by a DC-to-DC converter circuit using a 4-pin YX8018 integrated circuit gated oscillator and a resistor. The YX8018 is a gated oscillator which runs at 200 Khz driving an open drain NMOS switch with output on pin 1. The internal circuit of the component pulses a small inductor to step up the voltage to drive the LED. The larger circuit is composed of: The YX8018 connected to the LED with pin 1 going to the LED positive lead. Pin 2—ground, is connected to the LED negative lead. Pin 2 is also connected to the negative lead (leading to the magnesium rod). Pin 3 is unconnected. Pin 4 connects to the positive lead (leading to the copper cylinder). A 150Ω resistor bridges pins 1 and 4 of the YX8018 to complete the circuit on the PCB, which drives the LED.

The circuit with LED resides inside the waterproof housing, with the LED outfacing inside the plastic dome to be visible. The housing is clipped to an attachment point on the top of the plastic stake inserted into the soil and angled as desired to make the LED visible to the user so that they may judge its brightness and adjust the moisture of the soil medium accordingly based upon device readings.

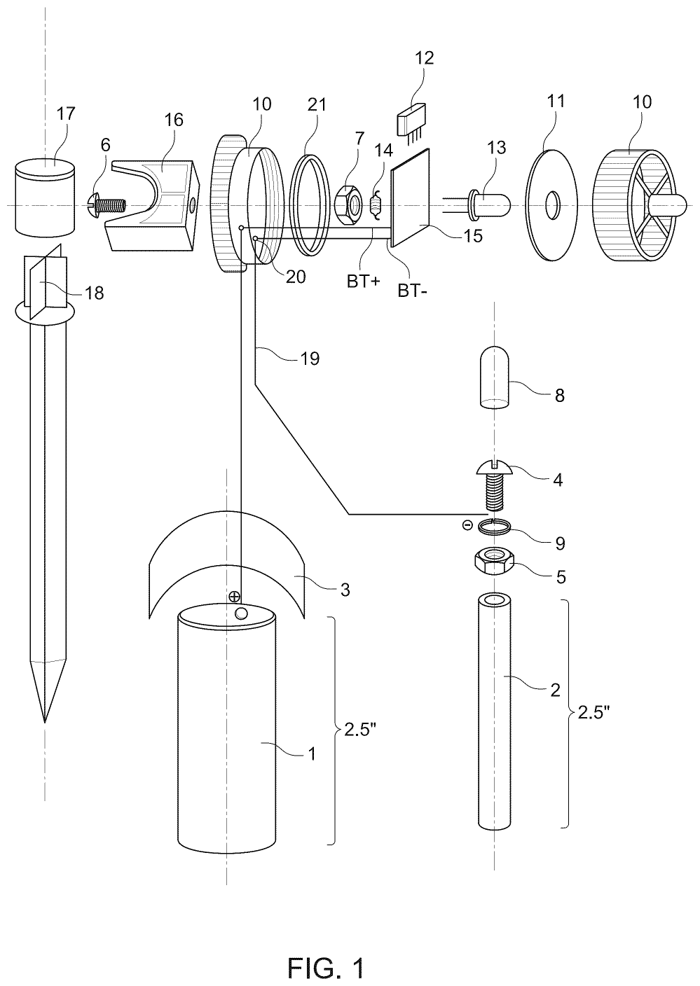

The present invention will now be described by referencing the appended figures representing a preferred, minimal embodiment. FIG. 1 depicts an example of a soil-powered LED plant water meter according to one possible embodiment of the present invention.

The 2½″ length of copper pipe (1) has a ⅛″ hole drilled into its side, ⅛ of an inch from the end. A 12″ length of 20-gauge wire (19), stripped at both ends feeds into the pipe at the end with the hole and is soldered to the pipe (1) at the hole. Black silicone rubber self-fusing tape (3) wraps around the very top ½″ of the pipe (1), covering the solder joint, and leaving the open part unobstructed. Clear silicone (20) is dabbed over the solder joint and exposed wire (19) on the interior of the pipe (1).

8-32¾″ stainless steel pan head machine screw (4) is screwed ½″ down into a 5/32 hole on the top side of the magnesium rod (2). Threaded onto the screw (4) is the 8-32 hex nut (5) with the lock washer (9) above it toward the head of the screw (4). 12″ of 20-gauge wire (19), stripped at both ends is connected to the rod (2), by being wrapped and cinched between the washer (9) and the tightened hex nut (5). The ⅜″ black screw thread protector (8) is placed over the screw (4) and wire connection, making seal with the magnesium rod (2).

Inside the 1″ round waterproof LED housing (10) sits the small PCB board (15). Its schematic is outlined in FIG. 2. Soldered to one end of the PCB (15) is the YX8018 4-pin gated oscillator (12). Connected to pins 1 and 4 is the 150Ω resistor (14). Pins 1 and 2 are connected to the green LED (13), which is bent to be parallel with the board (15). Pin 2 is also soldered to the negative power input, the 20-gauge wire (19) attached to the magnesium rod (2). Pin 4 is soldered to the positive power input, the 20-gauge wire attached to the copper pipe (1). Pins 3 and 4 are connected to each other, completing the circuit.

The PCB board (15) sits perpendicular to the housing, with the LED (13) pointing upward. The housing has 2 5/64 holes on the back for the two 20-gauge wires (19) comprising the positive and negative leads. Two dabs of clear silicone (20) seal the wires (19) with the housing (10) making a waterproof seal. The 6-32 screw (6) goes into the hole on the 20 mm plastic mounting bracket (16) and is fed through a 5/32 hole on the back of the housing (10) and threaded into the stop nut (7) which sits in the housing interior, to affix the bracket (16) to the housing (10). The ⅞″ diameter thin white plastic disk (11) sits around the hole for the LED, creating a backing to prevent light from filling the housing (10) interior. Both halves of the housing (10) are screwed together with the gasket (21) making a seal in the middle, and the LED (13) outfacing inside the clear plastic dome.

The ¾″ of 20 mm plastic tubing (17) sits on top of the plastic stake (18). The 20 mm plastic bracket (16) affixed to the back of the housing (10) snaps onto the tubing (17) on top of the stake (18), attaching the housing (10) to the stake (18) so that the wires (19) face downward.

In order for the device to function, the DC-DC converter circuit (FIG. 2) is absolutely necessary. Other 4-pin oscillator integrated circuits may be used instead of the YX8018, including the QXS521 and the YX8018b as long as resistor values are adjusted accordingly.

The copper cathode and magnesium anode yield the best results due to their differences on the galvanic scale, and the fact that the by-product of the sacrificial anode (MgO2) is not only not harmful to plants and soil, but is actually a beneficial nutrient which is essential in order for photosynthesis to occur. Different metals with differing corrosion potentials may be used to achieve basic functionality, but the same results are not guaranteed, and oxidation of other metals such as zinc or iron may contribute to toxicity of the soil medium and affect plant health negatively, and would not be recommended in a preferred embodiment of the current apparatus. Carbon and/or Graphene is a conductive and non-toxic metal that may be used for anode or cathode construction; however, it does not offer the same benefits as the time-release magnesium supplementation which occurs in the current embodiment of the device.

The copper cathode must be at least ¾″ wide with a length of 2+ inches in order to have enough surface area to be able to receive the electrons from the corroding magnesium through the soil medium in an efficient enough manner to generate the necessary current. A cathode of smaller diameter will not yield desirable results because it will be unable to pick up electrons properly and will impede device functionality.

Using a larger magnesium rod anode and a larger diameter copper cylinder cathode will increase the available current of the device to a point, however this will not improve functionality, as the LED is already achieving full brightness with the aforementioned setup and a further increase in current will decrease the device's sensitivity to moisture, making it less effective. Different shapes may be used for the anode and cathode such as flat plates, rods, cylinders and mesh with varying results. The current embodiment is ideal for portability, as the anode fits perfectly inside the cathode and plastic stake to make it compact and easily transportable for field testing.

The round green 3.3v LED is strongly recommended but not required. Different color, size and voltage-rated LEDs may be used to diminished visible effect and brightness with varying cutoff voltages. This will affect the moisture level at which the LED will shut off, effecting the accuracy of the device as a water meter but perhaps adding an ornamental or aesthetic quality. Green light is invisible to plants so it will not impact a plant's photoperiod at all in a fully-dark setting.

Different shape housings with various ornamental qualities may be used made out of different materials such as wood, metal and glass. Using a waterproof housing is recommended to protect the circuitry and wire connections from water used to water the soil medium.

Various mounting methods and hardware may be used to mount the waterproof LED housing in a visible way, such as clips to attach the housing to the edge of a pot or a user-added stake. The plastic stake is not necessary to achieve device functionality but it is used to achieve visibility and adjustable angling of the LED to enhance the user experience. The aforementioned stake could be made of any material such as wood, metal or glass and is not restricted to plastic construction.

Embodiments can include ornamental details such as statuary, miniature decorations, crystals, prisms and globes to cast the LED light, and the device housing may be oriented facing outwards, upwards, or upside down to achieve a desired lighting effect.

Different protective/waterproofing methods may be used to protect the wire connections on the anode and cathode such as silicone, waterproof heat-shrink and marine-grade terminal connections attached to the wire.

On the PCB, the QXS521 or YX8018b oscillator may be used instead of the YX8018, however the resistor value will have to change as well. A 330Ω resistor will need to be used with the QXS521 oscillator and a 150Ω resistor will need to be used with the YX8018b oscillator.

One possible assembly method of the preferred embodiment of the present invention described here is as follows:

Drill a ⅛″ wide hole just under the edge at one end of the copper pipe. Bore a ½″ deep hole with a 5/32 drill bit into the end of the magnesium rod. Screw the stainless steel screw into the hole in the rod with the nut and lock washer threaded onto the screw with the washer on top.

Drill a ⅛″ hole into the middle of the back of the waterproof housing to accommodate the clip attachment. Drill 2 holes symmetrically and horizontally opposite each other at the bottom of the back of the housing with a 5/64 drill bit to accommodate the positive and negative 20 gauge wires.

Place the lock nut over the inside of the hole in the middle of the back of the housing and feed a screw through the white plastic clip. Screw the screw through the back of the housing and secure it tightly into the lock nut on the inside, affixing the clip to the back of the housing with prongs facing out.

As described in FIG. 2, Solder the YX8018 IC to the small PCB board. Solder pin 1 to the positive lead of the LED. Solder pin 2 to the negative lead of the LED as well as the negative power connection. Solder pin 4 to the positive power connection. Solder a 150Ω resistor between pins 1 and 4. Power connections are made to 9″ lengths of 20-gauge wire.

Place the PCB board within the waterproof housing with the two power connections coming out the back through the pair of opposite holes in the bottom of the back of the housing with the negative connection on the left and the positive connection on the right. Dab the holes with clear silicone on the inside of the back of the housing to make a seal. Bend the LED so that it is centered and facing outward from the interior of the housing. Screw on the front of the housing with the white disk with the hole placed on the inside and the rubber gasket making a waterproof seal. Make sure that the LED is centered in the plastic dome on the front of the housing.

Strip the end of the positive power connection and solder it to the hole drilled into the copper cylinder with the wire going in through the inside of the cylinder. Strip the end of the negative power connection and wrap it around the screw going into the magnesium rod, cinching it tightly between the washer and the nut. Place the rubber cap over the screw and wire connection, making seal with the magnesium rod. Wrap and stretch a ½″ wide piece of silicone self-fusing tape over the side of the edge of the copper cylinder to conceal the wire connection.

Snap the small length of plastic tubing onto the top of the plastic stake. Snap the clip on the back of the housing onto the plastic tubing, securing it so that the wires face downward.

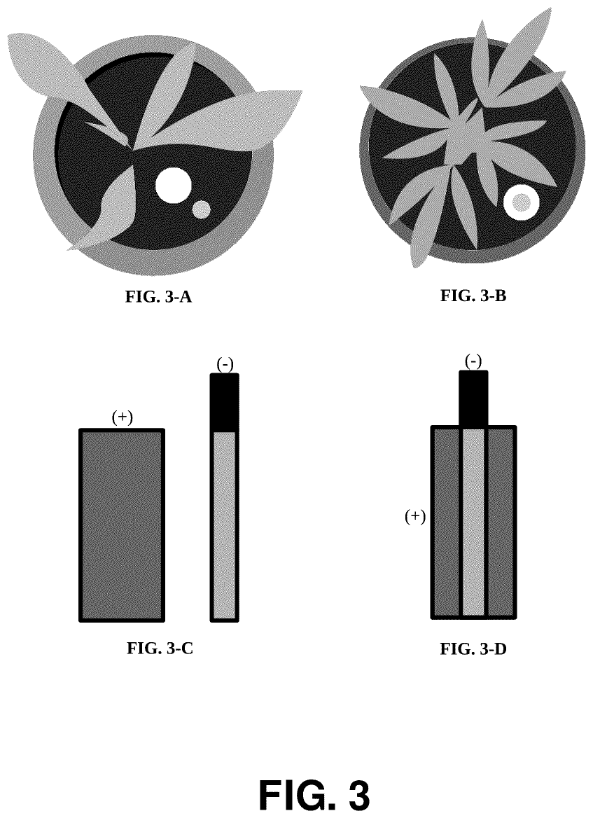

As depicted in FIG. 3, there are two possible configurations for setting up the present invention for full operation and function. Configuration A (FIG. 3-A) is for long-term setup in a soil medium with plant. Configuration B (FIG. 3-B) is for short-term use in a soil medium with plant or field testing of a soil medium. FIG. 3-C describes the anode and cathode orientation as viewed from the side for configuration A, with one directly next to the other separated by soil medium. FIG. 3-D describes the anode and cathode orientation as viewed from the side for configuration B, with one inside the other separated by soil medium.

Configuration A:

For long term installation insert soil from the base of the plant to be monitored into the bottom end of the copper cathode and depress it until it is more compact then the surrounding soil, there is no empty space inside the cylinder and the soil stays in the cylinder. Wet the soil very slightly to get it to stick together if it is very dry.

Dig a small hole the depth and width of the copper cylinder near the base of the plant and insert the cylinder into the hole so that its top is about even with the soil level. Or if the soil is loose enough, push the cylinder directly into the soil.

Make a small area of soil next to the cylinder on any chosen side about as compact as the soil inside the cylinder. This will be the location where the magnesium rod anode will be inserted into the soil next to the copper cylinder.

Insert the magnesium rod anode into the compact soil patch directly next to the copper cylinder so that the rubber cap is above the soil line. The rubber cap can be as close to the cylinder as touching it—without the magnesium touching the copper. At this point in the initial setup, the LED should be off or very dim.

Insert the plastic stake into the soil medium close to the anode/cathode site at the location desired to view the LED from. Clip the LED housing onto the top of the plastic stake and rotate and adjust as desired to best view the LED.

Proceed to water the plant to be monitored as you would normally and make sure that the anode/cathode site is as saturated as the base of the plant if it is a distance from the plant. Wetting the entire soil medium works best. The LED should fully illuminate indicating that the soil medium is saturated. If it does not illuminate, try configuration B.

Configuration B:

Configuration B works best for short term installations but in certain soils with very low electrical conductivity (EC), configuration B will be the best method for long term setup. It is also the preferred method for taking field test readings with the device.

Insert the magnesium anode directly into the middle of the moist soil-filled copper cathode so that the plastic cap on the top of the rod is on the same side as the silicon tape on the copper cathode and that the rod is straight and not contacting the walls of the copper cylinder.

If the moist soil is not compact enough, push more down into the cylinder to accommodate the rod, and top off the cylinder with soil on both ends once the rod is inserted if necessary, so that there is no empty space inside the cylinder.

Insert the cylinder back into the hole so that it is even with the soil level and make sure that the soil is compact surrounding the cylinder. The LED should now be illuminated indicating that the soil medium is saturated.

To dim the brightness of the device, pull the magnesium rod up and out of the soil slowly until the desired brightness is achieved.

As the soil medium dries, the LED will grow dimmer and eventually shut off indicating soil dryness. Depending on the watering needs of the plant being monitored, it will help the user know when to water the plant.

After a period of operation (2+ months), the magnesium anode may need to be cleaned with sandpaper or steel wool to remove magnesium oxide buildup if normal operation is impeded. After 4-6 months of normal operation, the magnesium anode may need to be replaced if it is badly corroded and not supplying current. Sanding the copper cylinder after a long period of operation (4-6 months) may improve performance but is not always necessary.

EC levels are affected by many factors and the remedies for enhancing EC improve the health of the soil as well. Soil amendments to improve soil conductivity include bio-char which provides a conductive path of carbon for electrons to flow more easily. Magnesium Sulfate added to water that is given to plants or soil will add salt which improves overall conductivity. The Magnesium Sulfate is also helpful in that it is able to erode the Magnesium Oxide passivation layer which forms on the surface of the magnesium anode over time as the galvanic reaction occurs. Blood meal will improve soil conductivity by adding more iron, a conductive electron path to the soil medium.

The water used on the soil medium or potted plant will directly impact the function of the device. Chlorinated water will decrease the EC of the soil as well as prematurely oxidize the magnesium anode. Water such as well water with suspended bicarbonates and minerals will cause and accelerate the formation of a passivation layer on the surface of the magnesium anode which will protect the Mg from oxidation, but will cut off its surface area with the soil medium, leading to a decline in device functionality. Distilled water has no conductive properties, so the device will not function in its presence without additional metals or minerals already present in the soil medium, or the addition of EC-boosting supplements like MgSO4, biochar or blood meal. Rainwater or reverse-osmosis filtered water is optimal for sustained device functionality but results will always vary due to differing minerals and metals present across a variety of different soil mediums.

Although the present invention has been illustrated and described herein with reference to a preferred embodiment and specific examples thereof, it will be readily apparent to those of ordinary skill in the art that other embodiments or examples may perform similar functions and/or achieve like results. All such equivalent embodiments and examples are within the spirit and scope of the present invention, are contemplated thereby, and are intended to be covered by the following claims.

What is claimed is:

1. An LED indicator light for the purpose of measuring the amount of moisture contained within a soil medium or soil medium containing a plant of any kind comprising:
a. A DC to DC converter circuit running an LED.
b. An anode of any size or shape consisting of magnesium or another metal with a high galvanic rating connected to the circuit's negative input.
c. A cathode of any size or shape consisting of copper or another metal with a low galvanic rating connected to the circuit's positive input.
d. A waterproof housing to contain the DC to DC converter circuit, LED and wire connections that renders the LED visible to the user.
e. A stake or method of attachment to position the waterproof housing such as clips or suspension to render the LED visible to the user.

2. The waterproof housing component of claim 1 further comprising any stylistic or aesthetic modifications that do not affect the basic function of the device.
